(19) United States
(12) Patent Application Publication
(10) Pub. No.: US 2012/0059485 A1
(43) Pub. Date: Mar. 8, 2012

(54) Total Knee Trochlear System

(75) Inventor: Gregory ROGER, St. Leonards (AU)

(73) Assignee: Advanced Surgical Design & Manufacture Ltd., St. Leonards (AU)

(21) Appl. No.: 13/043,699

(22) Filed: Mar. 9, 2011

(30) Foreign Application Priority Data
Mar. 9, 2010 (AU) 2010900966

(51) Int. Cl. A61F 2/38

(52) U.S. Cl. 623/20.35

(57) ABSTRACT

A method of performing a knee replacement procedure in an individual in need thereof. The method comprises preparing the distal end of the individual's femur to receive a femoral component of a knee prosthesis. The preparation includes resecting a proximal surface and a distal surface of a medial and/or lateral condyle of the femur, determining the amount of uncut bone left between said resected proximal and distal surfaces after said resection, and then determining, from the amount of uncut bone, a pre-operative, natural profile of a trochlear groove between the medial and lateral condyles of the individual's femur. A selection of femoral components can then be provided with the one selected preferably having medial and lateral condyle components and a trochlear section therebetween that has a profile which substantially matches the natural profile of the trochlear groove of the individual. Knee prostheses components and kits are also provided.

### CROSS-REFERENCE TO RELATED APPLICATIONS

The present application claims the benefit of Australian provisional application no 2010900966, filed Mar. 9, 2010, the content of which is incorporated herein by reference.

### FIELD OF THE INVENTION

The application is directed to artificial knee replacement.

### BACKGROUND

Total knee replacement is also referred to as a “tri-compartmental” knee replacement. The three compartments are the medial and lateral tibio-femoral articulation and the patello-femoral articulation.

The patello-femoral joint is the sliding, with some slight rotation, of the patella down the trochlear groove of the femur as the knee flexes. The patella is a sesamoid bone, invested in the quadriceps tendon which arises from the powerful quadriceps muscle and then after investing the patella inserts into the tibial tubercle of the proximal tibia.

The patella is also invested in, and to some degree constrained by, the retinaculum, a dense sheet of soft tissue attached to the femur medially and laterally and, with the patella and the quadriceps tendon forming an almost hemispherical cuff at the front of the femur. The Quad-Patella-Tendon complex is extensible which is how the knee flexes and extends, but the retinaculum is essentially inextensible unless stretched or torn or operatively divided.

The patello femoral articulation carries considerable load including the through-carried load of the quadriceps. Further, if the knee is flexed to 90 degrees and a person stands on one leg, the patello-femoral joint is loaded up to between 4 to 6 times body weight. Even at lesser degrees of flexion there is substantial load and this is why full extension is required to avoid chronic patella pain.

Problems that occur at this joint include mal-tracking of the patella. The angle made by the quadriceps, through the patella to the tibial insertion, the Q-Angle, is not 180 degrees, but a variable number less than this. This results in a “bow string” effect and the patella tends to want to slide laterally—more commonly in females. The patella is constrained from doing this by the retinaculum, the shape of the trochlear groove and the vastus medialis oblique muscle (VMO). On occasions the retinaculum must be released laterally and in more serious cases the insertion to the tibia shifted to stop this painful mal-tracking.

During normal movement, the patello-femoral articulation does not have any bearing on knee flexion, except when there is wear of the patella and flexion causes crunching noises and sometimes pain. Following joint replacement surgery, however, the patello-femoral joint can be instrumental in reducing flexion, may mal-track and also may “defunction” the quadriceps mechanism.

In implanting a knee replacement, if the situation is such that the patella implant is correctly placed and has the same thickness as the natural patella once it is installed, then it is clear that for any given point on the patello-femoral articulation, the natural articulation may be replicated by moving the femoral component proximally/distally as well as anteriorly or posteriorly. However, these movements of the femoral components have potentially undesirable impacts on the other functioning of the knee, the articulation with the tibia as well as the bone cuts required to place it there.

Because of this, surgeons currently typically place the femoral implant according to the tibio-femoral joint line and either the posterior condyle level or the anterior femoral cortex level, depending upon the particular systems philosophy. The resultant position of the patello-femoral tracking is therefore determined solely by the design of the femoral component, rather than desirably in response to variations of the individual patient's anatomy.

In practice it is observed that there are effectively as many femoral shapes as there are femurs. For any given joint design, the femoral component will largely match that patient's functional anatomy, perhaps as high as 60% of cases. For the rest, there need to be releases of soft tissue which are performed to accommodate the new femoral component's differences to the patient's original anatomy.

Where the femoral component stands too proud in the patello-femoral articulation (including the articulation of the retinaculum in the cheeks of the condyles) this area gets relatively very tight upon flexion and so the patient is slow to get full flexion, or might never achieve it. This is difficult to fully compensate for with soft tissue releases.

Similarly, if the implant is too low in the patello-femoral tracking the lever arm or pulley effect of the patella is reduced and so the effectiveness of the quads in extending the tibia is reduced. Patients experience this as a sense of weakness going down stairs. This is, however, less obvious, especially on the operating table, and so there has been a tendency to bias femoral component designs to this low profile distortion.

There is a need therefore to improve patello-femoral tracking during and post knee replacement surgery in addition to ensuring the proper articulation of the femur and the tibia relative to one another.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### SUMMARY

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

In one aspect, there is provided a method of performing a knee replacement procedure in an individual in need thereof; said method comprising:

preparing the distal end of the individual's femur to receive a femoral component of a knee prosthesis said preparation including resecting a proximal surface and a distal surface of a medial and/or lateral condyle of the femur;

determining the amount of uncut bone left between said resected proximal and distal surfaces after said resection;

determining, from the amount of uncut bone, a pre-operative, natural profile of a trochlear groove between said medial and lateral condyles of the individual's femur;

providing a selection of femoral components;

selecting a suitable femoral component having medial and lateral condyle components and a trochlear section therebetween;

wherein said trochlear section has a profile which substantially matches the natural profile of the trochlear groove of said individual.

The femoral components to be selected may each comprise the same condylar dimensions relative to one another and differ only in the depth of the trochlear section relative to an anterior surface of said condyles. The depth is measured from the anterior surface of the condylar components to the base of the recessed trochlear section.

The difference in depth of the trochlear sections in a kit of femoral components may comprise between about 1 mm and about 10 mm. Typically, the difference between the components is in the range of about 2 mm to about 6 mm. In one embodiment, the kit includes two femoral components wherein the depth of trochlear sections differ relative to one another by up to about 4 mm.

Where there is relatively little uncut bone between the resected surfaces this is indicative that the patient's patello-femoral tracking was, in a natural state, positioned relatively anteriorly or in other words sat quite “proud” in the knee joint. To re-create such an anteriorly positioned tracking between the patella and the trochlear section of the femoral component of the prosthesis, the present invention provides a selection of femoral components having differing trochlear section profiles. In this instance, the surgeon may choose to use a relatively less deep trochlear section to re-create the natural tracking.

In an embodiment where there is a relatively greater amount of uncut bone this can be taken as an indication that the patient's patello-femoral tracking had a relatively low profile. In this instance, the surgeon may choose to use a femoral component with a relatively deep trochlear section to re-create the lower profile tracking of the patella and the femur.

The surgeon may then select the correct cutting block for the “proud” or “low profile” femur and intra-operatively account for the patient's anatomy through use of a femoral component having a suitable trochlear groove shape and/or depth and thereby improve flexion reliability and quadriceps function for each patient.

In another aspect, the application is directed to a femoral component of a knee prosthesis mountable to the distal end of a patient's femur and comprising a bearing surface comprising a pair of condylar surfaces each having an anterior/posterior curvature, said pair of condylar surfaces defining therebetween an anterior trochlear groove engageable with a patella or patellar component, wherein the trochlear groove has a shape and/or depth to ensure the patella or patellar component is positionable to maximise flexion and quadriceps function of the patient.

In another aspect, the present invention is a method of implanting a knee prosthesis comprising at least a femoral component as defined herein, the method comprising:
measured the trochlear groove depth with reference to a fixed reference location;
preparing the distal end of the patient's femur to receive the femoral component;
assessing the prepared distal end to determine the patient's normal patello-femoral tracking;
selecting a femoral component having a trochlear groove having a shape and/or depth that ensures a patella or patellar component is engageably positioned relative to the trochlear groove to maximise the flexion and quadriceps function of the patient.

In a further embodiment, having selected a particular shape femoral component, with the best fit of trochlear groove depth, the method can comprise adjusting the total thickness of the patella through surgical resection of the articulating surface and resurfacing with a patella component of a known thickness.

In this embodiment, the difference between the amount of resected patella and the thickness of the patella component can comprise the amount of adjustment to the patella femoral tracking height at a given angle of flexion. Typically, this adjustment is optimised for the range of 60 to 90 degrees of flexion.

In one embodiment, the fixed reference location can comprise a temporarily implanted pin or a definitive anatomical point. The trochlear groove depth can be compared with the depth of the prosthesis trochlear groove once the size and shape of prosthesis is selected.

In one embodiment of the aspects, maximising quadriceps function can be understood as one approach to minimising the loss of the power of the quadriceps following total knee replacement.

To establish optimal patello-femoral tracking, the trochlear groove should be of a suitable depth to match the pre-operative depth of the groove. In addition to the dimension of depth, other factors may be considered including the proximal to distal placement of the femoral component as compared to the original joint line.

In another embodiment, a tracking curve of the trochlear groove which is related to the depth but also related to the shape of the curve may be varied.

In yet a further embodiment, the anterior-posterior positioning of the implant during a procedure may impact the patello-femoral tracking.

Further, the thickness of the patella implant as compared to the bone removed from the patella to install that implant will impact upon the alignment and relative movement of the patella and the trochlear groove.

The tracking depth of the patello-femoral joint at, for example 45 degrees, when compared to how the natural patello-femoral joint was placed can be determined by:
the shape of the natural patello-femoral joint for that patient;
the shape of the implant's patello-femoral joint; and
whether there was any proximal/distal or anterio/posterior offset of the implant in the surgical implantation.

Modification of positioning of the femoral component in a knee replacement to properly align the patella and the trochlear groove is undesirable as this would potentially impact on other functioning of the knee, the articulation with the tibia as well as the bone cuts required to place it there.

While other functions of the knee may dictate the orientation of the femoral component of a prosthesis, by modifying the profile of the trochlear section of a prosthetic implant, an optimal patello-femoral tracking may be achieved while still facilitating optimal orientation of the other components of a knee replacement assembly.

The femoral component as described herein therefore has a trochlear section size and/or shape that is selected in light of the patient's anatomy so that the resulting tibio-femoral articulation and the contact areas for the patient remain either the same or as close as possible to what the patient achieved through their natural anatomy.

In one embodiment, the width of the trochlear section of the femoral component may also be varied in light of the patient's anatomy. In another embodiment, the contours of the surface forming the trochlear section may be selected in light of the patient's anatomy.

In yet another embodiment, the trochlear section may be substantially constant along its length whereas in other embodiments, the trochlear section may vary in depth, width or shape along some, the majority, or all of its length.

In one embodiment, the knee prosthesis will comprise a tibial component in the form of a plate. The plate can be planar. The tibial component can also have at least its upper surface comprised of polished chromium alloy. The plate is preferably formed on its underside with fixation means such as pegs or screws adapted for insertion into undersized holes formed in the tibial plateau. The pegs serve to hold the tibial component in place on the tibia. If desired, other fasteners of the tibial component with the tibia could be used. While the upper surface of the tibial component can be planar it could be formed with grooves.

### BRIEF DESCRIPTION OF THE DRAWINGS

By way of example only, embodiments are now described with reference to the accompanying drawings:

FIG. 1 is an anterior view of one embodiment of a knee prosthesis with the patella and the patellar component of the prosthesis removed for clarity, the position of the patella being shown in broken lines; and

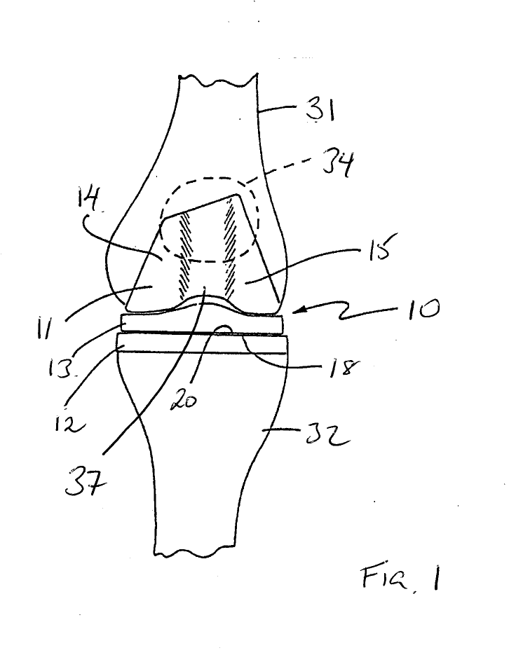

FIG. 2 is a section in an anterior-posterior plane through another embodiment of a knee prosthesis, with the patellar and patellar component present as shown.

### PREFERRED MODE

A method of performing a knee replacement procedure is defined herein. The method comprises:

preparing the distal end of the individual's femur to receive a femoral component of a knee prosthesis said preparation including resecting a proximal surface and a distal surface of a medial and/or lateral condyle of the femur;

determining the amount of uncut bone left between said resected proximal and distal surfaces after said resection;

determining, from the amount of uncut bone, a pre-operative, natural profile of a trochlear groove between said medial and lateral condyles of the individual's femur;

providing a selection of femoral components;

selecting a suitable femoral component having medial and lateral condyle components and a trochlear section therebetween;

wherein said trochlear section has a profile which substantially matches the natural profile of the trochlear groove of said individual.

An embodiment of a knee prosthesis that can be selected using the method defined herein is generally depicted as 10 in the Figures. While the depicted embodiment is one example of a knee prosthesis, it will be appreciated that other types of knee prosthesis can be inserted using the method of the current invention.

The knee prosthesis 10 comprises a femoral component 11, a tibial component 12 and between them a meniscal bearing 13.

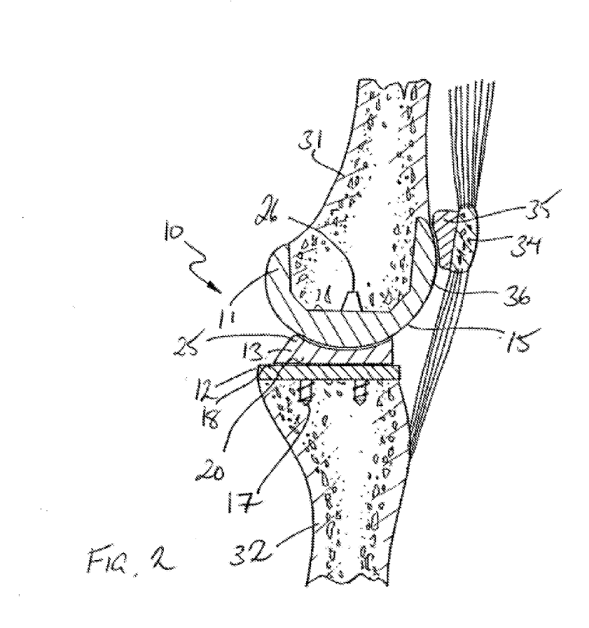

The femoral component 11 is formed of a suitable chromium alloy and is adapted to be attached to the distal end of the femur 31 through use of a pair of pins 26 together with a bone cement such as polymethyl methacrylate or other known methods. The femoral component 11 comprises an anterior flange 36 and a pair of substantially parallel condylar bearing surfaces 14 and 15. As seen in FIG. 2, the anterior flange 36 is adapted to slidingly engage with a patellar component 35 which is attached to the posterior side of the patellar 34. As the knee 10 is flexed, the patellar component 35 will move into contact with the trochlear groove 37 that separates the femoral condyles.

In the depicted embodiment, the tibial component 12 comprises a flat plate 16 having a pair of downwardly extending pins 17 on its underside to allow attachment of the component 12 to the proximal end of the tibia 32. The upper surface 18 of the depicted tibial component 12 is flat and smooth. The tibial component 12 is also formed of a suitable chromium alloy.

The depicted meniscal bearing 13 is formed of an ultra high molecular weight polyethylene. The meniscal bearing 13 can be U-shaped in this embodiment with the base of the U being directed anteriorly. The cut-out in the U-shaped meniscal bearing 13 can provide space for the posterior cruciate ligament if it is retained. If it is desired to accommodate both the anterior and posterior cruciate ligaments the cut-out would need to be deeper, or the bearing to be in two sections.

The underside 20 of the depicted meniscal bearing 13 is flat and smooth. It is adapted to slide smoothly over the upper surface 18 of the tibial component 12. The upper side of the meniscal bearing 13 can be provided with a pair of recesses. Each recess being adapted to receive one of the condylar bearing surfaces 14 and 15 of the femoral component 11. In a medio-lateral plane the recesses 21 are arcuate and correspond in shape with the substantially uniform shape of the condylar bearing surfaces 14 and 15 when sectioned medio-laterally. In an anterior-posterior plane each of the recesses can also be arcuate.

The meniscal component 13 can extend posteriorly beyond each of the recesses to provide a “heel” 25 to the meniscal component 13. The heel may extend posteriorly substantially horizontally from the back of each recess. Alternatively, the heel may slope downwardly from the back of each recess to meet the underside 20 as is seen in the embodiment depicted in FIG. 2. In either case, the heel 25 can help prevent the meniscal, bearing 13 from lifting at its anterior edge when the knee is in full flexion.

Irrespective of the type of knee prosthesis utilised in the invention, the anterior trochlear groove 37 is engageable with a patella or patellar component 35 (as depicted in FIG. 2). The trochlear groove 37 has a shape and/or depth to ensure the patella or patellar component is positionable to maximise flexion and quadriceps function of the patient.

Implantation of the knee prosthesis according to the present invention, such as the prosthesis 10 depicted, can comprise:
measuring the trochlear groove depth with reference to a fixed reference location;
preparing the distal end of the patient's femur 31 to receive a femoral component;
assessing the prepared distal end to determine the patient's normal patello-femoral tracking;
selecting a femoral component having a trochlear groove 37 having a shape and/or depth that ensures a patella or patellar component is engageably positioned relative to the trochlear groove to maximise the flexion and quadriceps function of the patient.

In selecting the femoral component, the components would not, however, vary in the tibio-femoral kinematics, allowing predictable flexion from this perspective.

In a further embodiment, having selected a particular shape femoral component, with the best fit of trochlear groove depth, the method can comprise adjusting the total thickness of the patella through surgical resection of the articulating surface and resurfacing with a patella component of a known thickness.

In this embodiment, the difference between the amount of resected patella and the thickness of the patella component can comprise the amount of adjustment to the patella femoral tracking height at a given angle of flexion. Typically, this adjustment is optimised for the range of 60 to 90 degrees of flexion.

In one embodiment, the fixed reference location can comprise a temporarily implanted pin or a definitive anatomical point. The trochlear groove depth can be compared with the depth of the prosthesis trochlear groove once the size and shape of prosthesis is selected.

Mal-tracking of the patella is thought to be primarily a function of the correct positioning of the femoral component, especially with reference to the internal/external rotation of the component on the femur as compared to the epicondylar axis. However, the current inventor considers that the depth of the trochlear groove 37 on the femoral component of a knee prosthesis alone or in combination with tightness of the PCL may also have some bearing on mal-tracking.

In the present invention, maximising quadriceps function can be understood as one approach to minimising the loss of the power of the quadriceps following total knee replacement.

In one embodiment, the depth of the trochlear groove 37 can relate to:

the designed depth of the groove with reference to the “cheeks” of the condyles and the position of the distal aspect of the condyles;

the proximal to distal placement of the femoral component as compared to the original joint line;

the designed tracking curve of the trochlear groove 37—related to the depth but also related to shape of the curve;

the anterior-posterior positioning of the implant; and/or

the thickness of the patella implant 35 as compared to the bone removed from the patella to install that implant.

In one embodiment, the thickness of the patella implant 35 compared to the bone removed can be substantially equal.

The tracking depth of the patello-femoral joint at, for example 45 degrees, when compared to how the natural patello-femoral joint was placed can be determined by:
the shape of the natural patello-femoral joint for that patient;
the shape of the implant's patello-femoral joint; and
whether there was any proximal/distal or anterio/posterior offset of the implant in the surgical implantation.

The femoral component as described herein has a trochlear groove size and/or shape that is selected in light of the patient's anatomy so that the resulting tibio femoral articulation and the contact areas for the patient remain either the same or as close as possible to what the patient achieved through their natural anatomy.

In one embodiment, the trochlear groove 37 profile including depth and width of the groove may be selected in light of the patient's anatomy.

In one embodiment, the trochlear groove 37 can have a depth that is the same as current prostheses, for example, the current “Active” prosthesis (Advanced Surgical Design & Manufacture Limited, Unit 2, 12 Frederick St, St Leonards, NSW 2065, Australia). In another embodiment, a knee prosthesis can be selected wherein the trochlear groove depth is 4 mm deeper than the “normal” prosthesis depth. In this embodiment, relatively more bone would be removed from the distal part of the front of the femur and the implant in that area would be lower down as viewed by the patella.

In yet another embodiment, the trochlear groove 37 can be substantially constant along its length whereas in other embodiments, the trochlear groove can vary in depth along some, the majority, or all of its length.

During an implantation procedure for a knee prosthesis, the anterior femoral cut and distal femoral cut could be made, as is usually done. The surgeon could then see how much bone is left untouched between the two cuts. Where there is relatively little uncut bone that indicates that this patient's patello-femoral tracking was relatively “proud”. Where there is relatively plenty of uncut bone this can be taken as an indication that the patient's patello-femoral tracking had a relatively low profile. The surgeon could then select the correct cutting block for the “proud” or “low profile” femur and intra-operatively account for the patient's anatomy through use of a femoral component having a suitable trochlear groove shape and/or depth and thereby improve flexion reliability and quadriceps function for each patient.

It will be appreciated by persons skilled in the art that numerous variations and/or modifications may be made to the invention as shown in the specific embodiments without departing from the scope of the invention as broadly described.

The present embodiments are, therefore, to be considered in all respects as illustrative and not restrictive.

1. A method of performing a knee replacement procedure in an individual in need thereof; said method comprising:
preparing the distal end of the individual's femur to receive a femoral component of a knee prosthesis said preparation including resecting a proximal surface and a distal surface of a medial and/or lateral condyle of the femur;
determining the amount of uncut bone left between said resected proximal and distal surfaces after said resection;
determining, from the amount of uncut bone, a pre-operative, natural profile of a trochlear groove between said medial and lateral condyles of the individual's femur;
providing a selection of femoral components;
selecting a suitable femoral component having medial and lateral condyle components and a trochlear section therebetween;
wherein said trochlear section has a profile which substantially matches the natural profile of the trochlear groove of said individual.

2. The method of claim 1 wherein the femoral components to be selected each comprise the same condylar dimensions relative to one another and differ in the depth of the trochlear section relative to an anterior surface of said condyles.

3. The method of claim 2 wherein the depth is measured from the anterior surface of the condylar components to the base of the recessed trochlear section.

4. The method of claim 3 wherein the difference in depth of the trochlear sections in a kit of femoral components for use in the procedure, comprises between 1 mm and 10 mm.

5. The method of claim 4 wherein the difference in depth between the femoral components is in the range of 2 mm to 6 mm.

6. The method of claim 5 wherein the kit includes two femoral components wherein the depth of trochlear sections differ relative to one another by up to 4 mm.

7. The method of claim 1 wherein a determination that there is a relatively reduced amount of uncut bone between the resected surfaces is indicative that the individual's pre-operative patello-femoral tracking was positioned relatively anteriorly in a knee joint.

8. The method of claim 1 wherein a determination that there is a relatively great amount of uncut bone between the resected surfaces is indicative that the individual's pre-operative patello-femoral tracking was positioned relatively inferior in a knee joint.

9. The method of claim 1 wherein the profile of the trochlear section of said femoral components is curved and wherein the curved profile may vary between said femoral components.

10. The method of claim 1 wherein the anterior-posterior positioning of the implant during the procedure may further change the tracking of the patella with the femoral component.

11. A femoral component of a knee prosthesis configured to be mounted to the distal end of a patient's femur, the femoral component having a bearing surface comprising:
a pair of condylar surfaces, wherein each condylar surface has an anterior/posterior curvature; and
an anterior trochlear groove between the pair of condylar surfaces,
wherein the anterior trochlear groove is configured to engage with a patella or patellar component such that the patella or patellar component can move its relative position so that the flexion and quadriceps function of the patient is essentially normal.

12. The femoral component of claim 11, wherein the anterior trochlear groove is configured to engage with a patella or patellar component based on the shape of the anterior trochlear groove.

13. The femoral component of claim 11, wherein the anterior trochlear groove is configured to engage with a patella or patellar component based on the depth of the anterior trochlear groove.

14. The femoral component of claim 11, wherein the anterior trochlear groove is configured to engage with a patella or patellar component based on both the shape and the depth of the anterior trochlear groove.

15. A knee replacement kit comprising:
a plurality of femoral components of a knee prosthesis configured to be mounted to the distal end of a patient's femur, wherein each of the plurality of femoral components has a bearing surface comprising:
a pair of condylar surfaces, wherein each condylar surface has an anterior/posterior curvature; and
an anterior trochlear groove between the pair of condylar surfaces,
wherein the anterior trochlear groove is configured to engage with a patella or patellar component such that the patella or patellar component can move its relative position so that the flexion and quadriceps function of the patient is essentially normal, and
wherein the plurality of femoral components have varying anterior trochlear grooves; and
a meniscal component of a knee prosthesis,
wherein each of the plurality of femoral components is configured to operatively engage with the meniscal component.

16. The knee replacement kit of claim 15, wherein the anterior trochlear grooves of the femoral components vary based on the shape of the grooves.

17. The knee replacement kit of claim 15, wherein the anterior trochlear grooves of the femoral components vary based on the depth of the grooves.

18. The knee replacement kit of claim 15, wherein the anterior trochlear grooves of the femoral components vary based on both the shape and the depth of the grooves.
